(19) United States
(12) Patent Application Publication
(10) Pub. No.: US 2008/0033335 A1
(43) Pub. Date: Feb. 7, 2008

(54) Methods and means for lifting jumper

(76) Inventors: Kendyl A. Roman, Sunnyvale, CA (US); Dean S. White, Belmont, CA (US); Joel P. Roman, Sunnyvale, CA (US)

Correspondence Address: KENDYL A ROMAN, 730 BARTEY COURT, SUNNYVALE, CA, 94087, US

(21) Appl. No.: 11/499,023

(22) Filed: Aug. 3, 2006

(51) Int. Cl. A61F 13/06; A63B 53/10; A61F 13/00

(52) U.S. Cl. 602/62; 473/317

(57) ABSTRACT

A leg sleeve with means for gripping used to aid in lifting a jumping player, especially for lineouts in Rugby Union. The simple, reuseable, easy to use leg sleeve provides a level of effectiveness, comfort, convenience, and safety not provided by conventional methods. Means for gripping include a thickened band or grip attached to a sleeve material. The means for gripping may be formed by attaching a semi-rigid material. The means for gripping may be formed by attaching extra layers in sections or in rolls. The means for gripping may be shaped to match the hand of a lifter. The means for gripping may comprise memory foam. The leg sleeve may further comprise a knee cutout or a hamstring cutout. To use, leg sleeves are applied to the legs of a jumper. When the jumper jumps, one or more lifters grip the leg sleeve. The lifters lift the jumper higher, support the jumper in the jump while the jumper attempts to intercept a ball, and safely lower the jumper.

### BACKGROUND

1. Field of the Invention

This invention relates to a leg sleeve, especially a leg sleeve with a means for gripping used when lifting player who is jumping, for example a jumper in a Rugby lineout.

2. Description of Prior Art

There is a need to jump high in order to intercept a ball in many sports, such as Rugby, Volleyball, or Baseball.

For example, in Rugby Union, when a ball goes out of bounds it is returned to play using a set formation known as a lineout. In a lineout, players from both teams line up near where the ball went out of bounds. Each team forms its own line. A space of about one yard is formed between the lines of players, referred to as the tunnel. The ball is then thrown back into the playing field. The ball must be thrown straight into the middle of the tunnel. Players compete for the ball. A player has a greater likelihood of winning the ball if he is higher in the air than others. Therefore, players jump and/or are lifted up by teammates to be in a favorable position to win possession of the ball.

Various methods have been employed to accomplish these types of lifts and jumps.

Originally, players jumped unassisted by teammates, but the rules of Rugby Union have changed to allow teammates to support a player while in the air. At first, lifters would grab the waist of the jumper. It is currently illegal to grab on to or bind to the player while the player is on the ground. As time passed, it became common to support a jumping player by grabbing the player by the bottom of the shorts and lifting him/her by the shorts long enough to play the ball. More recently, it has been made legal to grab the jumping player by the thighs above the knee. Many jumping players wrap a combination of materials and tapes around the thigh to offer those lifting a better grip.

The use of such techniques has several disadvantages such as:
Being ineffective, as a good grip is often still difficult to maintain
Being unsafe, as jumping players that are dropped because of poor grip can fall up to 10 feet and land in an awkward position
Damage to or tearing of clothing, especially if the proper shorts are not worn
Extreme discomfort to the jumper
Taking time to apply during the critical stages of pre-match preparation
Requiring help to apply, as taping one's own leg is often difficult to do satisfactorily
Needing to be taped before every game
Requiring special skill of the lifter in the case where only one lifter is used

It is also desirable to have a means for lifting that does not cause additional bunching and riding up of the short rugby shorts and that looks better than an awkward contraption of tapes on both thighs.

There is a need for a means to improve lifting in all levels of play, as ineffective lifts can be costly, dangerous, and contribute significantly to losing a game.

What is needed is a quick, simple, easy to use, resusable device that provides an effective means of obtaining a sure grip that offers comfort and safety to the players involved.

### SUMMARY OF THE INVENTION

Accordingly, it is an objective of the present invention to provide a quick, easy to use, simple, reusable, effective means of lifting a jumper that provides safety and comfort to those involved.

### Objects and Advantages

Accordingly, beside the objects and advantages described above, some additional objects and advantages of the present invention are:

### 1. To provide a comfortable method of lifting a player who is jumping

### 2. To provide a more effective method of obtaining a sure grip on the thighs of a jumping player

### 3. To provide an increase of safety for a jumping player being lifted by the thighs

### 4. To provide a method of lifting that is easy to use

### 5. To provide a method of lifting that is reusable

### 6. To provide a method of lifting that offers a higher performance

### 7. To provide a method of lifting that can be quickly applied and removed

### 8. To provide a method of lifting that is adjustable to varying conditions

### 9. To empower a less skilled lifter to lift a jumper by himself

### 10. To provide a method of lifting that is minimal in cost and waste

### 11. To provide a method of lifting that does not worsen a player's appearance

### 12. To provide a method of lifting that does not interfere with the wearing of compression shorts

### DRAWING FIGURES

In the drawings, closely related figures have the same number but different alphabetic suffixes.

FIG. 1A and FIG. 1B show prior art techniques for lifting a jumper.

FIG. 2 illustrates a leg sleeve with means for gripping.

FIG. 3A through FIG. 3C illustrate various embodiments of the means for gripping.

FIG. 4 shows the use of the present invention.

FIG. 5A through 5D illustrate various embodiments of the leg sleeve construction.

FIG. 6 illustrates the leg sleeves integrated into a pair of shorts.

### REFERENCE NUMERALS IN DRAWINGS

| | |
|---|---|
| 110 | first jumper |
| 112 | shorts |
| 120 | first lifter |
| 121 | left hand of first lifter |
| 122 | right hand of first lifter |
| 130 | second lifter |
| 131 | left hand of second lifter |
| 132 | right hand of second lifter |
| 140 | ball |
| 150 | second jumper |
| 154 | thigh |
| 160 | third lifter |
| 161 | left hand of third lifter |
| 162 | right hand of third lifter |
| 170 | fourth lifter |
| 171 | left hand of fourth lifter |
| 172 | right hand of forth lifter |
| 180 | third jumper |
| 190 | underwrap |
| 192 | (a-b) tape |
| 194 | bulge |
| 200 | (a-d) leg sleeve |
| 210 | means for gripping |
| 220 | attachment |
| 230 | sleeve material |
| 310 | semi-rigid material |
| 320 | covering layer |
| 330 | (a-b) extra layer |
| 350 | roll |
| 510 | knee cutout |
| 520 | hamstring cutout |
| 550 | (a-b) shaped grip |
| 560 | (a-b) memory foam |
| 570 | (a-b) impression |
| 580 | back grip |
| 590 | front grip |
| 600 | shorts with integrated sleeves |

### Special Definitions

sleeve—a substantially tubular part designed to fit over another object, such as a leg.

### DESCRIPTION OF THE INVENTION

The present invention comprises a leg sleeve comprising a sleeve material and a means for gripping. When a player jumps, one or more other players can assist the jumper by gripping the means for gripping. Such a lifter is able to lift the jumper higher, support the jumper while in the air, and safely lower the jumper to the ground. When the jumper is not jumping, the jumper is able to run or otherwise move without hinderance or discomfort.

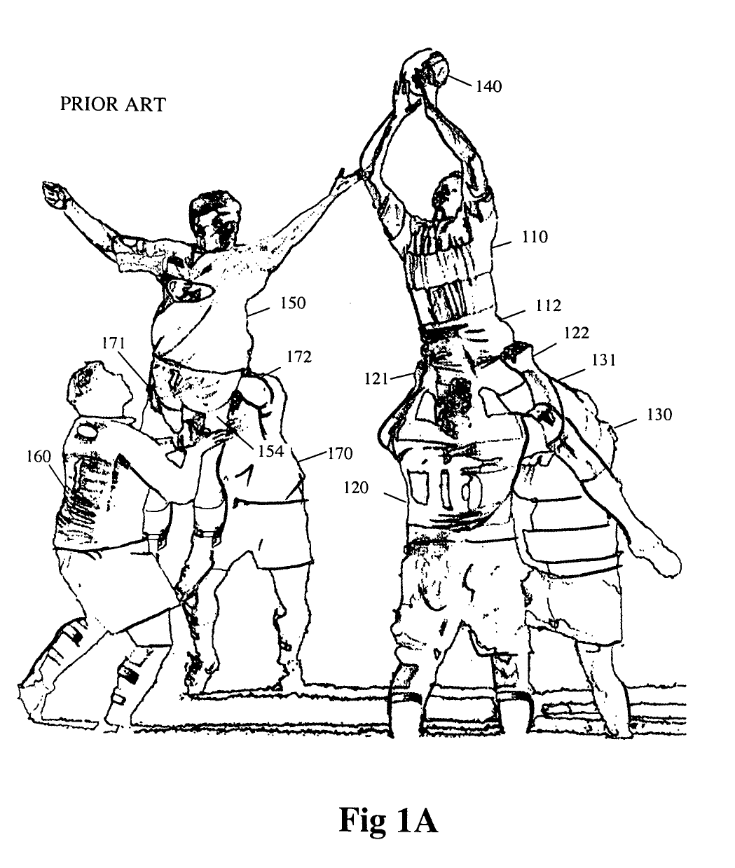

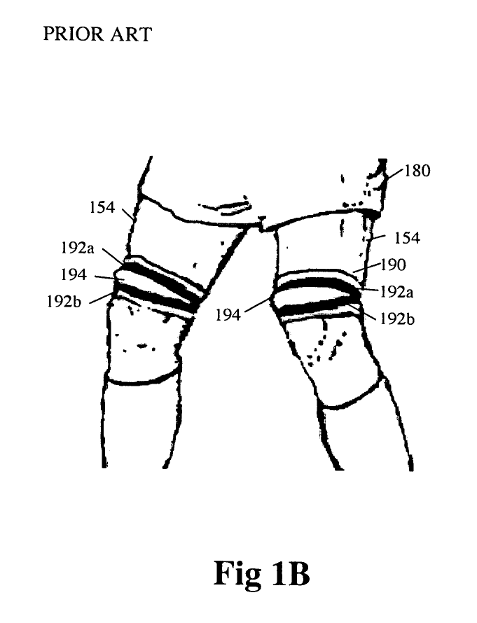

### FIG. 1A

FIG. 1A illustrates two conventional means of lifting. As shown on the right, a first jumper 110 is lifted by a first lifter 120 and a second lifter 130. As shown on the left, a second jumper 150 is lifted by a third lifter 160 and a fourth lifter 170.

A first conventional means for lifting (gripping the shorts) is shown on right. The first jumper 110 is lifted by his shorts 112. The first lifter 120 lifts using both his left hand 121 and his right hand 122 on the bottom front of the shorts 112. The second lifter 130 is gripping the shorts with his left hand 131 and his right hand 132 (not shown) to lift. These grips allow the first lifter 120 and second lifter 130 to lift first jumper 110 to a height where he can intercept the ball 140.

A second conventional means for lift (gripping the thigh) is shown on the left. The fourth lifter 170 is using a similar technique of lifting as the second lifter 130 and uses both his right hand 171 and his left hand 172. Third lifter 160 is lifting second jumper 150 by his thigh 154. Note that the second jumper 150 is unstable, is falling, and was unable to intercept the ball because of the bad lift.

Lifting by the shorts has many disadvantages including, for example, discomfort for the jumper, less than optimal lift performance, and difficult to obtain grip. It is also difficult to obtain a secure grip on a bare thigh (e.g. third lifter 160).

### FIG. 1B

FIG. 1B illustrates a third jumper 180 with material wrapped around his thighs 154. The one-time-use material consists of an underwrap 190 covered by an upper strip of tape 192a and a lower strip of tape 192b. The underwrap 190 may consist of fabric athletic tape, foam tape, or cloth. Typically the tape is black, plastic, electrical tape. This arrangement creates a bulge 194. The bulge 194 may include additional layers of the underwrap 190 material.

The one-time-use thigh wrap also has many disadvantages including, for example, difficulty in applying consistently, requiring application by coach or other player during the critical pre-game preparation time, discomfort, waste of materials, and debris often left behind.

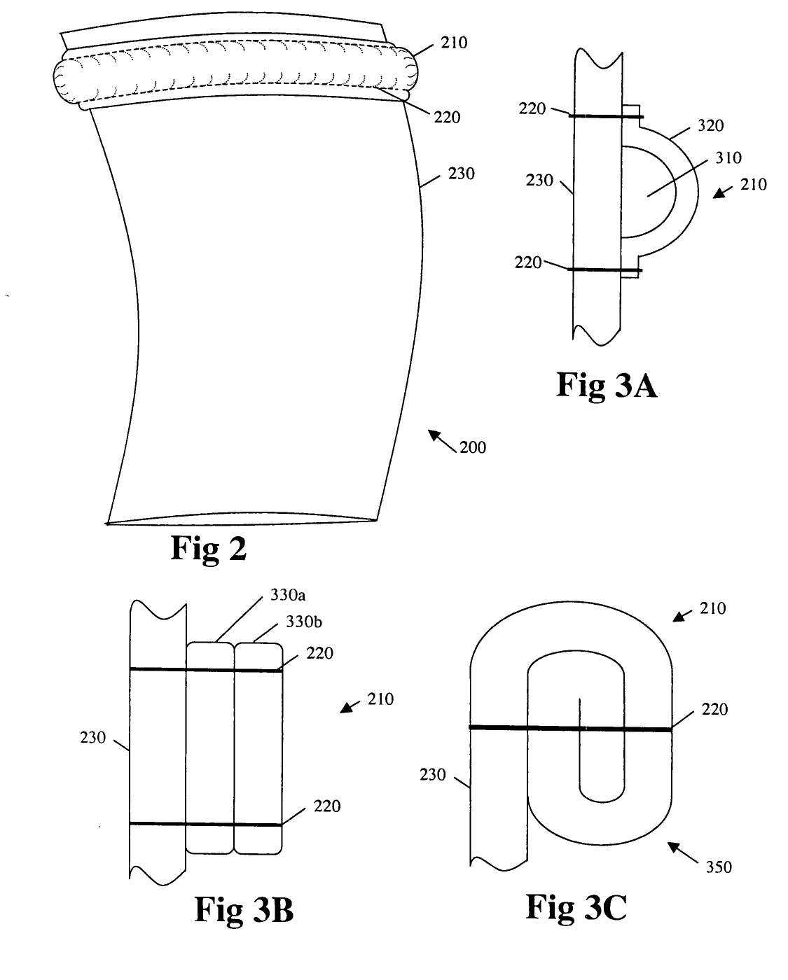

### FIG. 2

FIG. 2 illustrates a leg sleeve 200 comprising a means for gripping 210 attached to a sleeve material 230 with an attachment 220. The sleeve material 230 is preferably a thin, flexible, elastic, durable material such as neoprene or stretch fabric. The attachment 220 is made by sewing with thread (as shown) or by gluing with an adhesive (not shown).

One advantage of using a flexible, elastic material is that the position of the leg sleeve may be adjusted to meet the needs of a specific conditions, such as a particular play. Further, the leg sleeve can be quickly removed from one player and transferred to another player to accommodate substitutions.

### FIG. 3A

FIG. 3A shows a cross-sectional view of one embodiment of the means for gripping 210. In this embodiment, the means for gripping 210 is created by covering a semi-rigid material 310 with a covering layer 320. The covering layer 320 is attached to the base sleeve material 230 by an attachment 220. In this embodiment, the semi-rigid material 310 comprises a higher density material than the sleeve material 230, and may be rubber, soft flexible plastic, or high-density foam.

### FIG. 3B

FIG. 3B shows another embodiment of the means for gripping 210. In this embodiment, one or more extra layers 330 are attached to the base sleeve material 230 by one or more attachments 220. As shown, two extra layers 330a and 330b are attached by sewing with thread.

When the base sleeve material 230 is neoprene, the extra layers 330 can be made of the same material to simplify manufacturing. When the base sleeve material 230 is a stretch fabric, the extra layers 330 could be neoprene or similar material.

Although shown at the top, a leg sleeve 200 could have multiple thickened areas at the top, middle, and bottom of the sleeve material 230.

### FIG. 3C

FIG. 3C shows another embodiment of the means for gripping 210. In this embodiment, a section of sleeve material 230 is rolled to form a roll 350. The roll 350 is secured by an attachment 220, shown sewn together with thread.

Although shown rolled at the top, the roll 350 also could be formed somewhere in the middle of the sleeve material 230 by pinching the material in the middle and rolling the resulting fold. The roll 350 could be then be held by the attachment 220.

Further, a leg sleeve 200 could have multiple rolls 350 at the top, middle, and bottom of the sleeve material 230.

### Extra Layers Using S-Shaped Fold

In yet another embodiment (not shown), for each means for gripping 210 the sleeve material 230 is folded forming an S-shaped fold. The S-shaped fold is held with an attachment 220, such as being sewn with thread. The S-shape fold in effect creates two extra layers 330 from the same material as the sleeve material 230 (similar to FIG. 3B, but without having the cut the sleeve material 230) and produces a rounded edge (similar to the roll 350 shown FIG. 3C).

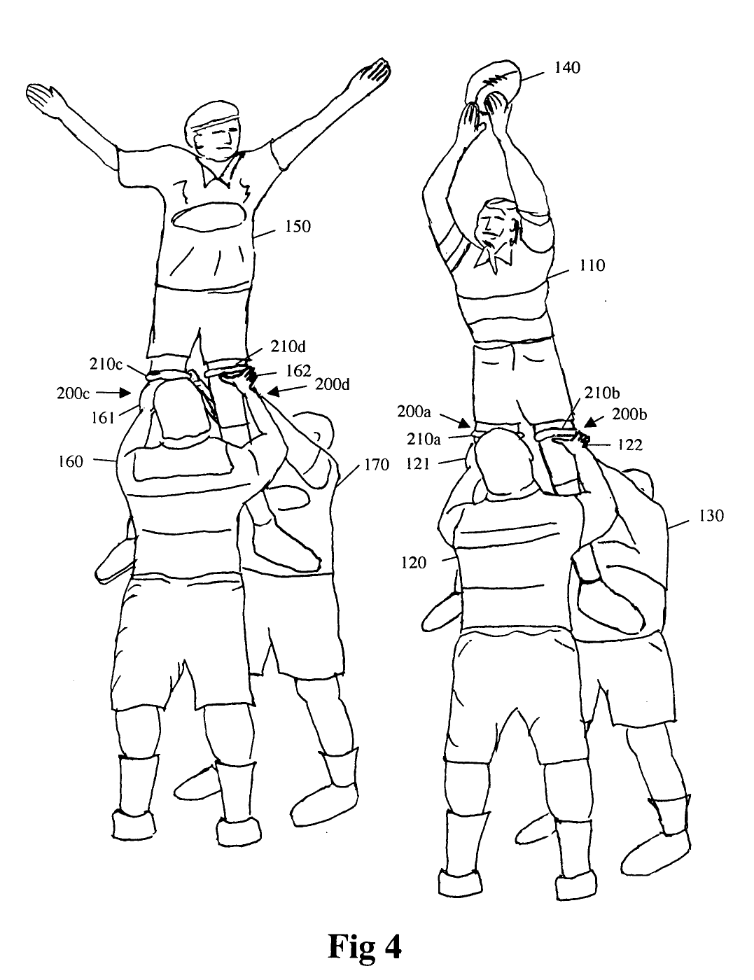

### FIG. 4

FIG. 4 shows leg sleeves 200a, 200b, 200c, and 200d, respectively, being used to assist in jumping and lifting for the lineout. Each leg sleeve 200 comprises at least one means for gripping 210.

Prior to entering the game, each potential jumper (e.g. first jumper 110 and second jumper 150) applies one or more leg sleeves 200 of the present invention to one or more legs. When an opportunity to intercept the ball occurs, each jumper jumps. One or more players from the jumper's team are then able to grip the leg sleeves 200. For example, as shown in FIG. 4, the first jumper 110 jumps during a lineout. The first lifter 120 places his hands (121 and 122) on the back of each leg sleeve 200a and 200b, respectively. The second lifter 130 places his hands (131 and 132) on the front of each leg sleeve 200a and 200b, respectively. Together, the first lifter 120 and the second lifter 130 are able to: a) lift the first jumper 110 higher, b) sustain the first jumper 110 at the height of the jump until the first jumper 110 is able to intercept the ball 140, and c) lower the first jumper 110 safely to the ground. Likewise, the third lifter 160 and the fourth lifter 170 are able to secure lift, sustain, and lower the second jumper 150 (shown wearing leg sleeves 200c and 200d, respectively), who is able to have a more competitive and safe attempt at the ball 140.

Because the respective lifters can obtain a safe and secure grip on the leg sleeves 200 of their respective jumper, both jumpers are able to: a) achieve a higher performance jump, b) be held stable during the jump, c) have a more consistent, more competitive attempt for the ball 140. The lifters are able to get a more consistent grip on the jumper. The jumper is able to jump with and be supported with less discomfort and more safety and confidence.

Compare the height and stability of the jumpers in FIG. 1A to FIG. 4 to see the increase in performance that may result from use of the present invention.

The increased safety and performance of the present invention may allow for changes in the Laws (rules) of Rugby to allow lifters to support jumpers in other situations, such as intercepting kicked balls, or block kicks.

Further, while the leg sleeves 200 of the present invention are shown as applied to Rugby, the leg sleeves could be used in other sports that also require jumping, such as Volleyball (e.g. while spiking a set ball, etc.) or Baseball (while jumping at the fence to stop a home run, etc.).

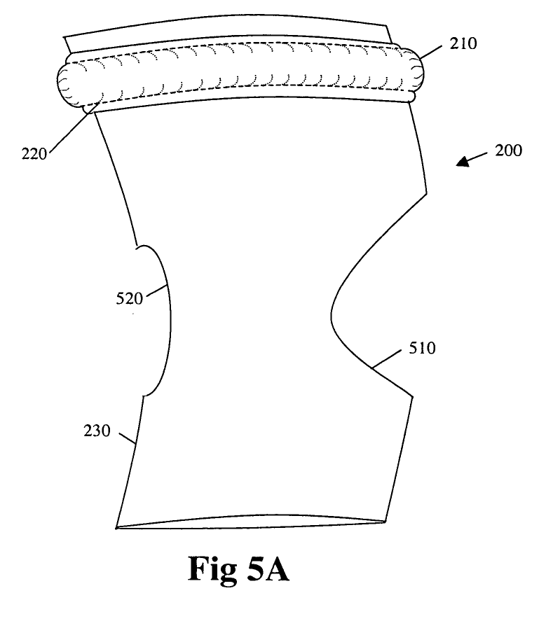

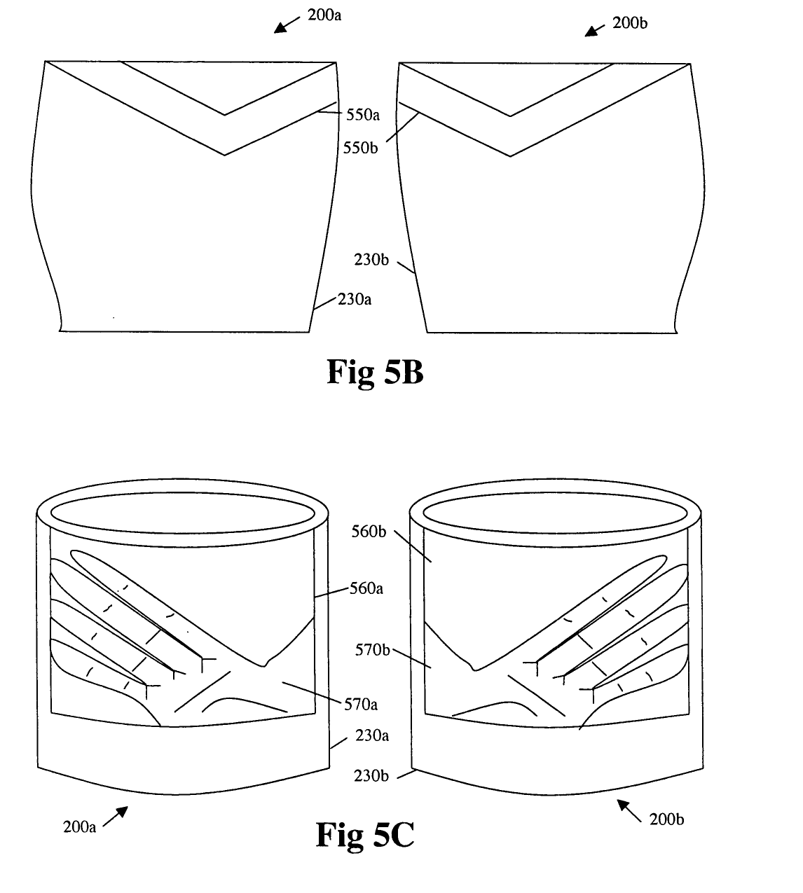

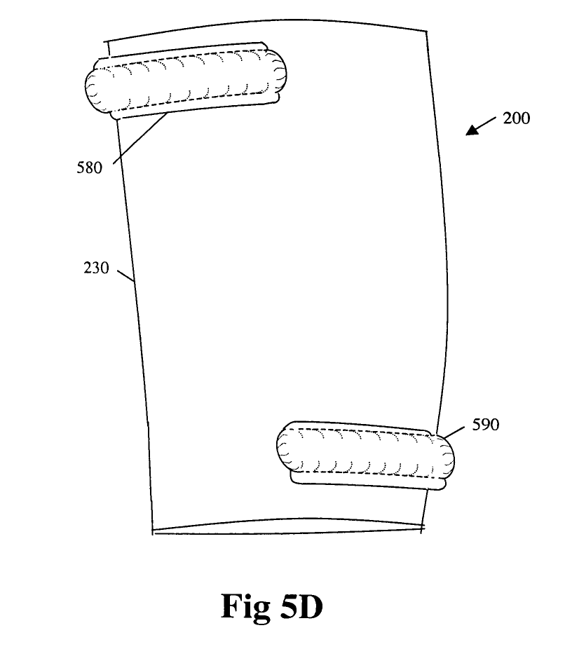

### FIG. 5A

FIG. 5A shows another embodiment of the leg sleeve 200 comprising a means for gripping 210 attached to the sleeve material 230 by the attachment 220. This embodiment further comprises an optional knee cutout 510 or an optional hamstring cutout 520 for greater comfort and flexibility. This embodiment may be worn in a position over the knee and provide additional knee support.

### FIG. 5B

FIG. 5B shows additional embodiments of leg sleeves 200a and 200b. The means for gripping are shaped grips 550a and 550b. Leg sleeve 200a comprises the shaped grip 550a attached to the sleeve material 230a. Leg sleeve 200b comprises the shaped grip 550b attached to the sleeve material 230b. The shape of each shaped grip 550 is designed to more closely fit the shape of the hand while gripping the thighs. For example, the shaped grip 550 may be designed to match the edge of extended thumb and extended index finger of each hand.

For example, in Rugby, because it is not legal to bind with the jumper before the jump is started, the lifters must assess a location to grip while the jumper is in the air. Thus, using the present invention, it is easier to place the open hands against the leg sleeves 200 until the palms of the hands rest against the shaped grips 550. In this way the lifter can quickly assess the location of at least one of the means for gripping 210 and immediately apply lifting pressure against the means for gripping 210.

### FIG. 5C

FIG. 5C shows additional embodiments of leg sleeves 200a and 200b. In this embodiment the means for gripping 210 comprise memory foam 560a and 560b attached to the sleeve materials 230a and 230b, respectively. The memory foam 560 conforms to the lifter's hands during contact, leaving impressions 570a and 570b. Regardless of where the lifter's hands make contact with the memory foam 560, the impressions 570 provide a immediate sure grip and the lifter can immediately apply a lifting pressure against the leg sleeves 200.

Because of the immediate, sure grip provided by the memory foam 560, the lifter does not have to spend time assessing the location of the means for gripping 210. In Rugby, because it is not legal to bind with the jumper before the jump is started, with this embodiment, the lifter may place the open hands against the leg sleeves 200 making an immediate impression. In this way the lifter can immediately apply lifting pressure against the means for gripping 210.

### FIG. 5D

FIG. 5D shows an additional embodiment of leg sleeve 200. Attached to the sleeve material 230 is a back grip 580 and front grip 590.

This embodiment provides a means for a single, relatively unskilled lifter to assist a jumper. For example, the single lifter can stand behind the jumper in a lineout. When the jumper jumps, the lifter is able to place his hands on the back of each leg sleeve 200 below the back grip 580. The jumper temporarily sits on the uplifted hands of the lifter. The back grip 580 prevents the lifters hands from sliding up the legs of the jumper. Having a single lifter allows for more offensive players to be distributed across the field and, under the rules of Rugby, reduces the number of defenders who can be close to the lineout.

This embodiment can also be used with two lifters, where a lifter's hand is placed on the back of the leg sleeve 200 against the back grip 580 and another hand is placed on the front of the leg sleeve 200 against the front grip 590 to provide a safe, secure, high performance lift.

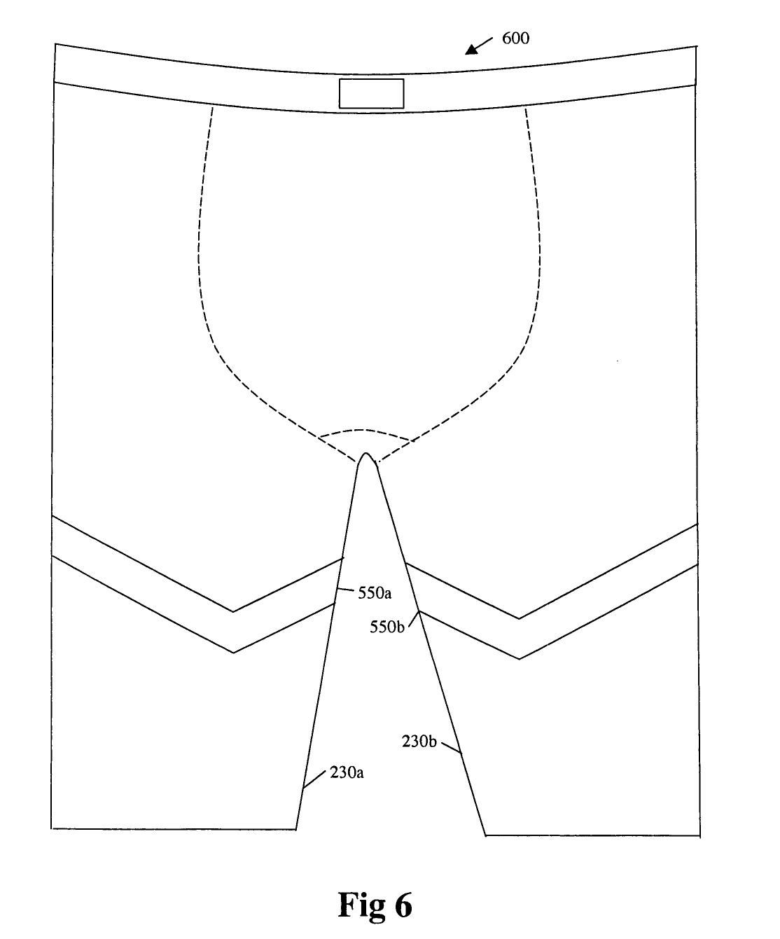

### FIG. 6

FIG. 6 illustrates a pair of shorts with integrated sleeves 600. This embodiment comprises a pair of shorts 600 having a right leg sleeve material 230a and a left leg sleeve material 230b integrated into the shorts 600. In this embodiment the means for gripping 210 is shown as shaped grips 550a and 550b, respectively, but could comprise any means of gripping 210. The shorts may be made of thin neoprene or other flexible, elastic material.

### Other Uses

While the descriptions of the various embodiments have been made in reference to Rugby Union, the present invention could also be used for other sports which involve, or in the future may involve, lifting, such as Volleyball, International Rules Football, Australian Rules Football, Baseball, or other sports.

### Advantages

### Comfortable

The leg sleeve of the present invention offers comfort to the jumping player. The sleeve does not apply pressure to the thigh or the sensitive crotch areas of the body, as conventional methods of lifting do. The wearer also does not risk pulling out the hairs of the leg during removal, as in certain conventional methods.

### Effective

The leg sleeve of the present invention is effective. A lifter has much better odds for obtaining and maintaining a sure grip with the present invention than with conventional methods. Thus, the jumper is more consistently able to obtain an optimum jump.

### Safe

Because of the effectiveness of the the present invention in allowing lifters to have a sure grip, the present invention offers greater safety and lessens risk of injury to both jumping and lifting players.

### Simple

The present invention is simple to make and use. The present invention requires little time to put on.

### Easy to Use

The present invention is easy to use. To install, the potential jumper simply pulls one sleeve over each leg. The lifters easily can find a grip while lifting the jumper.

Unlike conventional methods of lifting, which require a second party such as a trainer or coach to apply and runs the risk of inconsistency, the present invention is easily put on by one person and gives consistent results.

### Reuseable

The present invention can be used over and over again. The conventional method of wraps and tapes can only be used once and a new one must be used for every game.

Because the conventional wraps and tapes can only be used once, the materials become trash after use. The remains of the wraps and tape are often strewn on the field and leave an unsightly, unprofessional appearance requiring extra effort to clean up.

### Higher Performance

The present invention offers higher performance in jumping than conventional methods. The quality of grip maintained allows stronger and higher lift, for example during Rugby lineouts. Use of the present invention gives teams more options on plays to be run during the lineout. The use of the present invention does not hinder agility or running, as conventional taping sometimes does.

### Quick

The present invention can be quickly applied and removed. Time from warm-up and pre-game training need not be taken to apply the leg sleeve. The conventional method of taping can take several minutes to apply and removal can also be a slow process. Conventional taping often requires assistance from a coach or other highly skilled person and detracts from the critical pre-game preparation.

### Adjustable

The present invention can easily be adjusted to different heights and positions on the leg. This easy adjustment enables the sleeve to be set to accommodate different lifters and/or different plays. The present invention can easily be switched from player to player to accommodate substitutions.

### Empowering

The present invention can empower a single player to lift jumper when that player would not be able to otherwise do so. This can offer teams a considerable advantage in the lineout as well as the reception of kicks.

### Efficient

The conventional method of taping is wasteful. Garbage is created after every game, and more taping and wrap materials need to be replaced and bought, adding to expenses. The present invention does not produce additional garbage or cost on repeated uses.

### Better Appearance

Conventional methods of lifting worsen the look of players. The present invention has a smooth, professional look. While convential taping has limited options, the present invention can easily be manufactured in a variety of colors to match the team color or the individual preference of the wearer. Space on the sleeve material may also be used to bear a team logo, national insignia, or advertising.

### Can be Used with Compression Shorts

Some players elect to wear compression shorts while playing their sport. Conventional methods of taping are ineffective when used with compression shorts. The present invention enables players to wear compression shorts and in combination with means for lifting.

### Cost Effective

Because the leg sleeve can be made relatively inexpensively, is reusable, and eliminates waste the present invention is cost effective.

### CONCLUSION, RAMIFICATION, AND SCOPE

Accordingly, the present invention provides an easy to use, simple, safe, comfortable, reusable, and effective means for lifting a jumper.

While the above descriptions contain several specifics these should not be construed as limitations on the scope of the invention, but rather as examples of some of the preferred embodiments thereof. Many other variations are possible. For example, the leg sleeves could be made of different materials or have additional features, or be used in different sports, without departing from the scope and spirit of the novel features of the present invention.

Accordingly, the scope of the invention should be determined not by the illustrated embodiments, but by the appended claims and their legal equivalents.

We claim:

1. A leg sleeve to be worn on a leg of a jumper and gripped by one or more lifters, said leg sleeve comprising:
a) sleeve material, and
b) a means of gripping, attached to said sleeve material with an attachment,
wherein the sleeve material comprises a thin, flexible, elastic material of sufficient size and shape to be worn around at least a portion of the leg of the jumper,
wherein, to apply the leg sleeve, the sleeve material is stretched and passed over the foot of the jumper and when positioned, in a predetermined position on the leg of the jumper, and released, said sleeve material compresses and holds the leg sleeve in the predetermined position while the jumper is running or jumping,
wherein the means of gripping is positioned such that, when the jumper jumps, the one or more lifters is able to grip said means of gripping with at least one hand,
wherein the means of gripping is of sufficient size and shape that it is easily gripped by the at least one hand of at least one lifter,
wherein, when the jumper jumps to obtain a higher position, the jumper is lifted and held in the higher position by the one or more lifters,
wherein, to remove, the sleeve material is stretched and passed over the foot and off the leg, and
wherein, to reuse, the sleeve material is stretched again,
whereby the jumper when wearing the leg sleeve is lifted and held in the higher position by at least one lifter.

2. The leg sleeve of claim 1 wherein the means of gripping comprises a thickened portion.

3. The leg sleeve of claim 2 wherein the thickened portion comprises a band that encircles the leg.

4. The leg sleeve of claim 2 wherein the thickened portion comprises,
i) a length of semi-rigid material which is firmer than the sleeve material, and
ii) a covering layer which covers the semi-rigid material,
wherein the semi-rigid material provides a firm area for the hand of at least one lifter to grip.

5. The leg sleeve of claim 2 wherein the thickened portion comprises one or more layers of material attached to the sleeve material,
wherein the one or more layers of material is substantially the same type of material as the sleeve material having the ability to stretch in substantially the same way that the sleeve material stretches, and
wherein the one or more layers protrudes outward from the base sleeve material and on at least the lower side of the protrusion provides an abrupt edge for the hand of the at least one lifter to grip,
whereby the jumper is able to:
i) stretch the sleeve material and the thickened portion when applying or removing the leg sleeve,
ii) run and jump free of discomfort or interference from the leg sleeve, and
iii) be supported by at least one lifter when jumping to intercept a ball, and
whereby the lifter is able to:
iv) readily ascertain the location of the thickened portion while the jumper is jumping, and
v) obtain a firm grip on the leg sleeve while lifting, supporting, and lowering the jumper.

6. The leg sleeve of claim 2 wherein the thickened portion comprises a section of the sleeve material which has been rolled into a roll of material and permanently held by the attachment,
wherein the roll having the ability to stretch in substantially the same way that the sleeve material stretches, and
wherein the roll of material protrudes outward from the base sleeve material and on at least the lower side of the roll provides a convex edge for the hand of the at least one lifter to grip.
whereby the jumper is able to:
i) stretch the sleeve material and the thickened portion when applying or removing the leg sleeve,
ii) run and jump free of discomfort or interference from the leg sleeve, and
iii) be supported by at least one lifter when jumping to intercept a ball, and
whereby the lifter is able to:
iv) readily ascertain the location of the thickened portion while the jumper is jumping, and
v) obtain a firm grip on the leg sleeve while lifting, supporting, and lowering the jumper.

7. The leg sleeve of claim 1 wherein the predetermined position on the leg extends from above a knee on the leg and below the knee, said leg sleeve further comprising a knee brace,
wherein the knee brace provides support for the knee joint,
whereby the knee brace provides the jumper with additional knee joint support and protection while running and jumping.

8. The leg sleeve of claim 1 wherein the predetermined position on the leg extends from above a knee on the leg and below the knee, said leg sleeve further comprising a knee cutout,
whereby the jumper is able to run and jump free of discomfort or interference from the leg sleeve covering the knee.

9. The leg sleeve of claim 1 wherein the predetermined position on the leg extends from above a knee on the leg and below the knee, said leg sleeve further comprising a hamstring cutout,
whereby the jumper is able to run and jump free of discomfort or interference from the leg sleeve covering the knee.

10. The leg sleeve of claim 1 wherein the means of gripping comprises a layer of memory foam of sufficient size and shape to match a substantial portion of a hand of one of the at least one lifters,
wherein when the hand makes contact with memory foam, the memory foam forms a temporary impression of the portion of the hand, and
wherein the impression provides a firm grip for the hand regardless of the position of the hand,
whereby the lifter is able to obtain a firm grip on the leg sleeve without having to first ascertain the location of the means for gripping.

11. A system for supporting a jumper by one or more lifters, said system comprising two leg sleeves in accordance with claim 1, said two leg sleeves comprising a first leg sleeve to be worn on a one leg of the jumper and a second leg sleeve to be worn on the other leg of the jumper,
wherein, prior to jumping, the first and second leg sleeves are applied to the corresponding legs of the jumper, and
wherein, when jumping, a first hand of the at least one lifter grips the means of gripping of the first leg sleeve and a second hand of the at least one lifter grips the means of gripping of the second leg sleeve,
whereby when the jumper jumps to higher position the at least one lifter is able to support the jumper with the first hand on the first leg sleeve and the second hand on the second leg sleeve.

12. The system of claim 11,
wherein the means for gripping of the first leg sleeve is shaped to match an extended thumb and an index finger of the first hand, and
wherein the means for gripping of the second leg sleeve is shaped to match an extended thumb and an index finger of the second hand,
whereby the lifter is able to obtain a firm grip on each of the leg sleeves when making contact with the respective leg sleeve after the jumper has already started to jump and is able to maintain the firm grip during the subsequent lifting, supporting, and lowering phases of the jump.

13. The leg sleeve of claim 1, wherein the means for gripping is positioned on the front of the leg.

14. The leg sleeve of claim 1, wherein the means for gripping is positioned on the back of the leg.

15. A system for supporting a jumper by one lifter, said system comprising two leg sleeves in accordance with claim 14, said two leg sleeves comprising a first leg sleeve to be worn on a one leg of the jumper and a second leg sleeve to be worn on the other leg of the jumper,
wherein prior to jumping the jumper is positioned with the back of the jumper towards the lifter and after jumping to the higher position, the lifter grips the means of gripping on the back of each leg sleeve,
whereby a single lifter is able to safely and effectively lift a jumper.

16. A method for supporting a jumper by one or more lifters comprising the steps of:
a) applying two of the leg sleeves of claim 1 to the legs of the jumper,
b) the jumper jumping into the higher position,
c) the one or more lifters ascertaining the location of each means of gripping,
d) the one or more lifters gripping a means of gripping with each hand,
e) lifting the jumper to a second higher position,
f) holding the jumper firmly in the second higher position until the jumper is able to intercept the ball, and
g) lowering the jumper safely and securely,
h) repeating steps b) through g) for a predetermined period of time,
i) removing the leg sleeves,
j) reapplying the leg sleeves, and
k) repeating steps b) through g) for a second predetermined period of time.

17. The leg sleeve of claim 1, wherein the lifters are consistently able to obtain a firm grip on the leg sleeve,
whereby the lifters are able to lift the jumper higher,
whereby the lifters are able to provide a more stable support for the jumper,
whereby the jumper is able to intercept the ball more often, and whereby the jumper is able to jump, be supported, and be lowered more safely,

18. The leg sleeve of claim 1, wherein a lifting force from the lifters is applied to the thighs of the jumper and wherein the lifting force is applied through the cushion of the sleeve material,
whereby the jumper experiences less discomfort and less injury.

19. A method for supporting a jumper by one or more lifters comprising the steps of:
a) applying two leg sleeves to the legs of the jumper, each leg sleeve comprising a means of gripping,
b) the jumper jumping into a higher position,
c) the one or more lifters gripping one of the means of gripping with each hand,
d) lifting the jumper to a second higher position,
e) holding the jumper firmly in the second higher position until the jumper is able to intercept the ball,
f) lowering the jumper,
g) repeating steps b) through f) at least once,
h) removing the leg sleeves,
i) reapplying the leg sleeves, and
j) repeating steps b) through f) at least once.

20. The system of claim 11,
wherein the first leg sleeve and the second leg sleeve are integrated into a pair of shorts.
